(19) United States
(12) Patent Application Publication
(10) Pub. No.: US 2025/0198925 A1
(43) Pub. Date: Jun. 19, 2025

(54) BODIPY DERIVATIVE COMPOUND AND FLUORESCENT PROBE FOR DETECTING CHANGES IN PH AND VISCOSITY OF OIL COMPRISING SAME

(71) Applicants: INSTITUTE FOR BASIC SCIENCE, Daejeon (KR); POSTECH Research and Business development Foundation, Pohang-si, Gyeongsangbuk-do (KR)

(72) Inventors: Young-Tae CHANG, Busan (KR); Xiao LIU, Pohang-si (KR); Eun Gyeong CHOI, Daegu (KR); Songbin CUI, Seoul (KR)

(73) Assignees: INSTITUTE FOR BASIC SCIENCE, Daejeon (KR); POSTECH Research and Business development Foundation, Pohang-si, Gyeongsangbuk-do (KR)

(21) Appl. No.: 18/845,716

(22) PCT Filed: Mar. 10, 2023
(86) PCT No.: PCT/KR2023/003296

§ 371 Date: Sep. 10, 2024

(30) Foreign Application Priority Data
Mar. 10, 2022 (KR) 10-2022-0030141
Mar. 9, 2023 (KR) 10-2023-0031046

(51) Int. Cl. G01N 21/64; C07F 5/02; G01N 33/03

(52) U.S. Cl. CPC G01N 21/6428; C07F 5/022; G01N 33/03; G01N 2021/6439

(57) ABSTRACT

Embodiment of the present disclosure relates to a BODIPY derivative compound and a fluorescent probe for detecting changes in the pH and/or viscosity of an oil including same. Since a fluorescent probe according to one embodiment can quickly detect a harmful oil with high sensitivity, it is possible to monitor the presence of harmful oils and/or cooking time, and determine whether waste oils have been mixed therein and/or the quality of the waste oils. In addition, a bad oil sensing system (BOSS) built by using the fluorescent probe is useful due to having portability and universal applicability in the field.

### TECHNICAL FIELD

The present disclosure relates to a BODIPY derivative compound, and a fluorescent probe for sensing changes in pH and/or viscosity of oil, comprising the compound.

### BACKGROUND ART

A cooking oil which is mainly made of glycerols or esters such as aliphatic acids produces various harmful materials such as trans fatty acids, peroxides, acrylamides, polycyclic aromatic hydrocarbons (PAHs), and polymer components, when heated. 120 million tons or more of cooking oil are being consumed worldwide every year, and some harmful cooking oils are illegally recycled and sold back to consumers or sold as a mixture with a fresh oil.

Cooking oil which has been used in cooking and is inedible is collected from drainage, frying oil, residual oil from animal fat, and the like, and is in a state of comprising by-products produced during the cooking process and being contaminated with heavy metal. Long-term consumption of the harmful cooking oil is known to be related to many diseases such as cardiovascular diseases, cancers, and Alzheimer's disease.

In order to solve a health problem due to consumption of inedible oil, various detection methods for detecting harmful oil have been developed, and there are two representative methods as follows.

First, a method of chemically detecting exogenous contamination or endogenous toxic by-products, and second, a method of detecting changes in physical properties such as density accumulated during cooking process, electrostatic capacity, and density (C. Zhu et al., RSC Adv. 2019, 9, 18285-18291). These detection methods comprise chromatography, mass cytometry, NMR, FTIR, Raman spectroscopy, and atom absorption spectroscopy, but these methods take lots of time and comprise preparation procedures, expert skills, expensive equipment, and complicated data analysis which are generally easily accessible to the public.

### DISCLOSURE

### Technical Problem

An object of the present disclosure is to provide a novel 4,4-difluoro-4-bora-3a,4a-diaza-s-indacene, C₉H₇BF₂N₂ (BODIPY) derivative compound.

Another object of the present disclosure is to provide a fluorescent probe for sensing changes in pH and/or viscosity of oil comprising the BODIPY compound.

Another object of the present disclosure is to provide a method of determining pH and/or viscosity of oil or sensing changes in pH and/or viscosity of oil, using the fluorescent probe.

Another object of the present disclosure is to provide a kit for detecting pH and/or viscosity of oil, comprising the fluorescent probe.

Another object of the present disclosure is to provide a device for detecting pH and/or viscosity of oil, comprising the fluorescent probe.

Still another object of the present disclosure is to provide a system for monitoring changes in pH and/or viscosity of oil, comprising the fluorescent probe.

### Technical Solution

In one general aspect, a compound represented by the following Chemical Formula 1 or a hydrate thereof is provided:

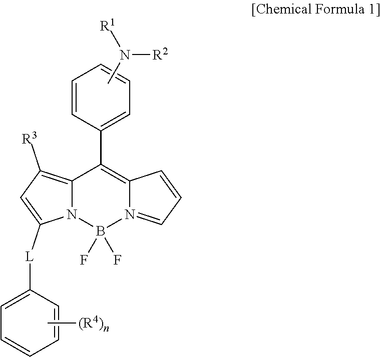

wherein
R¹ and R² are independently of each other —H, —C(O)H, C₁₋₁₀ alkyl, or C₁₋₁₀ alkylcarbonyl;
R³ is —H or C₁₋₅ alkyl;
R⁴ is independently of each other —H, —OH, —NH₂, C₁₋₈ alkyl, C₁₋₈ alkoxy, C₁₋₈ alkylamino, or C₁₋₈ alkylcarbonyloxy;
n is an integer of 1 to 3; and
L is C₂₋₁₀ alkenylene.

In an example embodiment, R¹ and R² may be independently of each other —H, —C(O)H, C₁₋₅ alkyl, or C₁₋₅ alkylcarbonyl.

In an example embodiment, R¹ and R² may be independently of each other —H or —C(O)CH₃.

In an example embodiment, R⁴ may be independently of each other —H, —OH, —NH₂, C₁₋₅ alkoxy, or C₁₋₅ alkylamino; n may be 1 or 2; and L may be C₂₋₅ alkenylene.

In an example embodiment, R⁴ may be independently of each other —H, —OH, or —OCH₃.

In an example embodiment, the compound represented by Chemical Formula 1 may be H

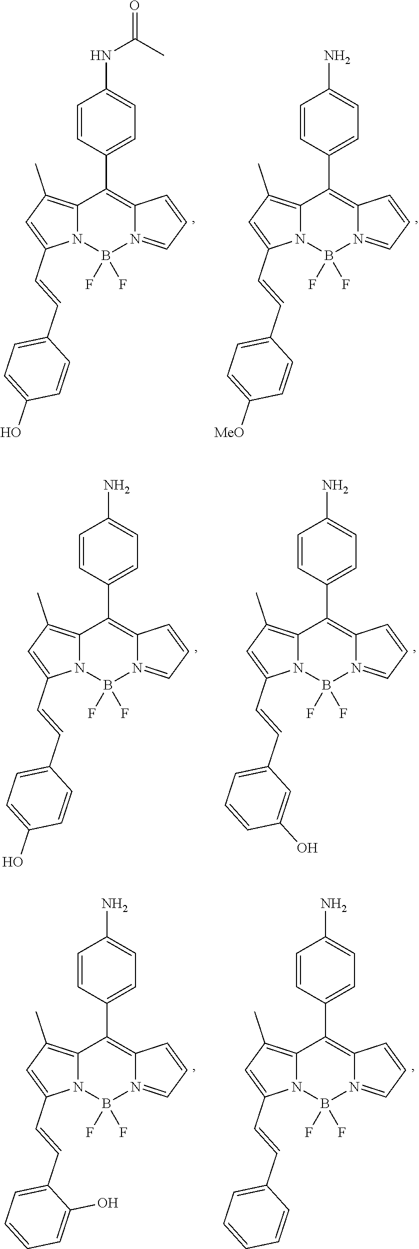

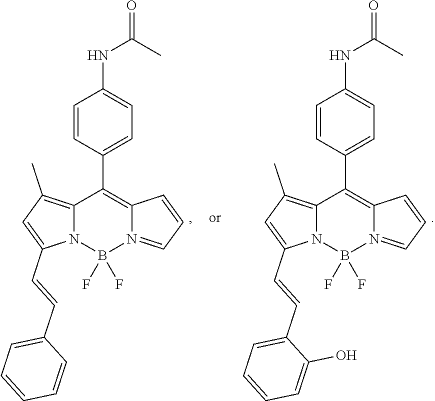

In another general aspect, a fluorescent probe for sensing changes in pH and/or viscosity of oil comprises the compound represented by Chemical Formula 1 or the hydrate thereof.

In an example embodiment, R¹ and R² may be independently of each other —H, —C(O)H, C₁₋₅ alkyl, or C₁₋₅ alkylcarbonyl.

In an example embodiment, R¹ and R² may be independently of each other —H or —C(O)CH₃.

In an example embodiment, R⁴ may be independently of each other —H, —OH, —NH₂, C₁₋₅ alkoxy, or C₁₋₅ alkylamino; n may be 1 or 2; and L may be C₂₋₅ alkenylene.

In an example embodiment, R⁴ may be independently of each other —H, —OH, or —OCH₃.

In an example embodiment, the compound represented by Chemical Formula 1 may be

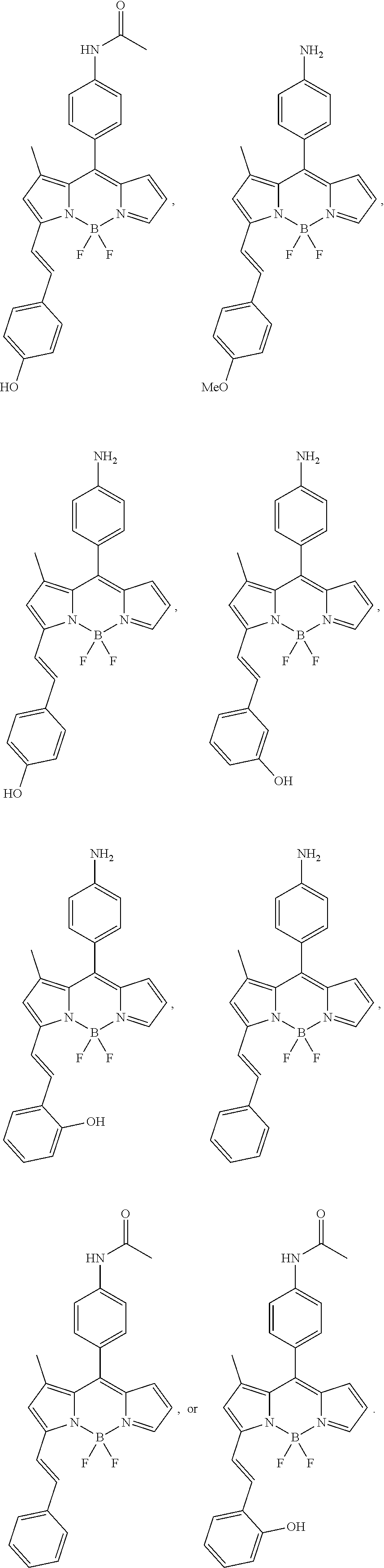

In another general aspect, a method of sensing changes in pH and/or viscosity of oil, using the fluorescent probe is provided.

In an example embodiment, the method of sensing changes in pH and/or viscosity of oil may comprise: mixing the fluorescent probe and the oil; and measuring a fluorescence intensity.

In another general aspect, a kit for detecting pH and/or viscosity of oil comprises the fluorescent probe.

In another general aspect, a device for detecting pH and/or viscosity of oil comprises the fluorescent probe.

In still another general aspect, a system for monitoring changes in pH and/or viscosity of oil comprises the fluorescent probe.

### Advantageous Effects

The present disclosure relates to a BODIPY derivative compound and a fluorescent probe for sensing changes in the pH and/or viscosity of oil, comprising the compound, and since the fluorescent probe according to an example embodiment allows rapid sensing of a harmful oil with high sensitivity, the presence or absence of harmful oils and/or cooking time may be monitored, and whether waste oil is mixed and/or waste oil quality may be checked. In addition, a bad oil sensing system (BOSS) built using the fluorescent probe is useful due to its good portability and universal applicability in the field.

### DESCRIPTION OF DRAWINGS

FIG. 1 is a photograph of a fresh oil (cooking oil) (Oil-1) prepared for the experiment, a 24 h-air-bubbling oil (Oil-2) which was continuously stirred while injecting air without heating, a 24 h-N₂-cooking oil (Oil-3) which was heated after filling a vial having a cap with a nitrogen (N₂) gas, and a 24 h-air-cooking oil (Oil-4) which was heated while injecting air.

FIG. 2 is a drawing showing the results of analyzing fluorescence intensities when Oil-1 and Oil-4 were treated with the compounds of Examples 1 to 8 and Comparative Example 1, respectively. The drawing inserted in the upper right corner of FIG. 2 is a photograph taken after treating the compound of Example 5 with Oil-1 (left) and Oil-4 (right), respectively.

FIG. 3 is a drawing showing the results of analyzing a fluorescence reaction of the compound of Example 5 in each cooking time when cooking with a cooking oil while supplying air and when cooking with a cooking oil under nitrogen gas, in order to analyze a fluorescence intensity depending on cooking time.

FIG. 4 is a drawing showing the results of analyzing a fluorescence reaction of the compound of Example 5 in mixed oils of a fresh oil and a harmful oil at each ratio, in order to analyze a fluorescence intensity depending on the amount of harmful cooking oil.

FIG. 5 is a drawing showing the results of analyzing a fluorescence reaction of the compound of Example 5 by preparing soybean oil (Soybean, 100%, OTTOGI Corp.), olive oil (Olive, 100%, CJ CHEILJEDANG CORP.), canola oil (Canola, 100%, CJ CHEILJEDANG CORP.), grapeseed oil (Grapeseed, 100%, CJ CHEILJEDANG CORP.), crispy cooking oil (Crispiness, 50% of canola oil and 49.8% of soybean oil, CJ CHEILJEDANG CORP.), and sunflower oil (Sunflower, 100%, HAEPYO) as fresh cooking oils before cooking (black bar) and those heated for 24 hours while injecting air (blue bars), in order to check reactions with the various cooking oils.

FIGS. 6 and 7 are drawings showing the results of checking viscosity and pH sensitivity (reactivity) of the compound of Example 5, respectively.

FIG. 8 is a drawing showing the results of analyzing emission ratios in cooking oil under various conditions, using viscosity-insensitive/pH-sensitive compound F1, in order to confirm relative contribution of pH to the reaction of the compound of Example 5.

FIG. 9 is a drawing showing the results of estimating pH changes using the results obtained in FIG. 8.

FIG. 10 is a drawing showing the results of analyzing viscosity depending on a mixing ratio and cooking time of castor oil in a mixture, after preparing the mixture of a fresh cooking oil and a castor oil as a viscosity model system, in order to confirm relative contribution of viscosity to the reaction of the compound of Example 5.

FIG. 11 is a drawing showing the results of analyzing a fluorescence intensity of the compound of Example 5 depending on the mixing ratio of the castor oil in the mixture of a fresh cooking oil and a castor oil.

FIG. 12 is a photograph of a bad oil sensing system (BOSS).

FIG. 13 is the results of measuring signals depending on cooking time of soybean oil (Soybean, 100%, OTTOGI Corp.), olive oil (Olive, 100%, CJ CHEILJEDANG CORP.), canola oil (Canola, 100%, CJ CHEILJEDANG CORP.), grapeseed oil (Grapeseed, 100%, CJ CHEILJEDANG CORP.), crispy cooking oil (Crispiness, 50% of canola oil and 49.8% of soybean oil, CJ CHEILJEDANG CORP.), and sunflower oil (Sunflower, 100%, HAEPYO), using BOSS.

### BEST MODE

The embodiments described in the present specification may be modified in many different forms, and the technology according to one embodiment is not limited to the embodiments set forth herein. In addition, the embodiments of an example embodiment are provided so that the present disclosure will be described in more detail to a person with ordinary skill in the art. Furthermore, throughout the specification, unless explicitly described to the contrary, “comprising” any constituent elements will be understood to imply further inclusion of other constituent elements rather than exclusion of other constituent elements.

The numerical range used in the present specification comprises all values within the range comprising the lower limit and the upper limit, increments logically derived in a form and spanning in a defined range, all double limited values, and all possible combinations of the upper limit and the lower limit in the numerical range defined in different forms. As an example, when it is defined that a content of a composition is 10% to 80% or 20% to 50%, it should be interpreted that a numerical range of 10% to 50% or 50% to 80% is also described in the specification of the present specification. Unless otherwise defined in the present specification, values which may be outside a numerical range due to experimental error or rounding off of a value are also comprised in the defined numerical range.

Hereinafter, unless otherwise particularly defined in the present specification, “about” may be considered as a value within 30%, 25%, 20%, 15%, 10%, or 5% of a stated value.

An example embodiment provides a compound represented by the following Chemical Formula 1 or a hydrate thereof, which is a 4,4-difluoro-4-bora-3a,4a-diaza-s-indacene (BODIPY, C₉H₇BF₂N₂) derivative;

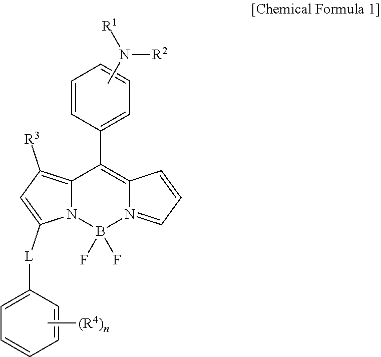

wherein
R¹ and R² are independently of each other —H, —C(O)H, C₁₋₁₀ alkyl, or C₁₋₁₀ alkylcarbonyl;
R³ is —H or C₁₋₅ alkyl;
R⁴ is independently of each other —H, —OH, —NH₂, C₁₋₈ alkyl, C₁₋₈ alkoxy, C₁₋₈ alkylamino, or C₁₋₈ alkylcarbonyloxy;
n is an integer of 1 to 3; and
L is C₂₋₁₀ alkenylene.

In an example embodiment, the alkenylene may comprise alkenylene directly connected to unsaturated carbon or alkenylene in which unsaturated carbon intervenes in and is connected to alkylene.

In an example embodiment, the “alkyl” comprises straight chain or branched chain alkyl, and also, those substituted by a substituent (for example, a halogen atom) which may be easily substituted by a person skilled in the art.

In an example embodiment, R¹ and R² may be independently of each other —H, —C(O) H, C₁₋₆ alkyl, C₁₋₆ alkyl, C₁₋₅ alkyl, C₁₋₃ alkyl, C₁₋₂ alkyl, —CH₃, C₁₋₈ alkylcarbonyl, C₁₋₆ alkylcarbonyl, C₁₋₅ alkylcarbonyl, C₁₋₃ alkylcarbonyl, C₁₋₂ alkylcarbonyl, or —C(O)CH₃.

In an example embodiment, R³ may be —H, C₁₋₅ alkyl, C₁₋₄ alkyl, C₁₋₃ alkyl, C₁₋₂ alkyl, or —CH₃.

In an example embodiment, R⁴ may be independently of each other —H, —OH, —NH₂, C₁₋₁₀ alkoxy, C₁₋₈ alkoxy, C₁₋₆ alkoxy, C₁₋₈ alkoxy, C₁₋₃ alkoxy, C₁₋₂ alkoxy, —OCH₃, C₁₋₁₀ alkylamino, C₁₋₈ alkylamino, C₁₋₆ alkylamino, C₁₋₅ alkylamino, C₁₋₃ alkylamino, C₁₋₂ alkylamino, or —NHCH₃.

In an example embodiment, n may be 1 or 2.

In an example embodiment, L may be C₂₋₅ alkenylene, C₂₋₄ alkenylene, C₂₋₃ alkenylene, or —CH═CH—.

In an example embodiment, when R⁴ is not —H, the compound may be ortho-, para-, or meta-substituted.

In an example embodiment, R¹ may be substituted at a meso-compound position, that is, at a para position. That is, the compound represented by Chemical Formula 1 may be a compound represented by the following Chemical Formula 2:

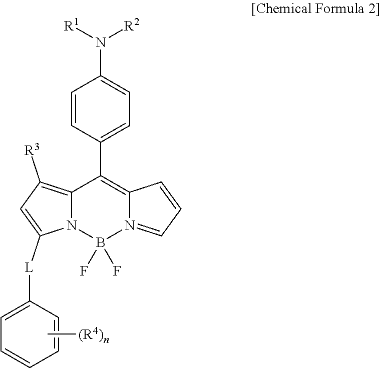

wherein R¹, R², R³, R⁴, n, and L are as defined above in Chemical Formula 1.

In an example embodiment, the compound represented by Chemical Formula 1 may be, specifically, for example,

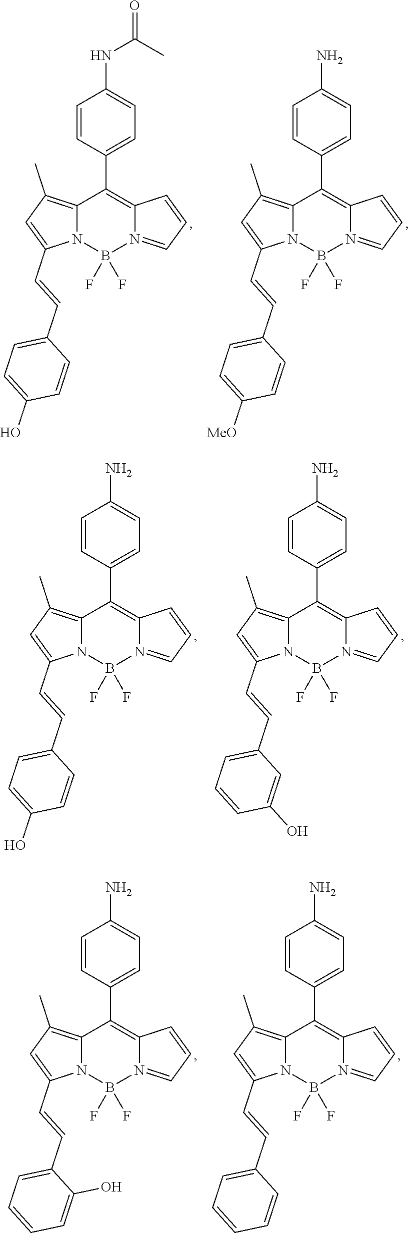

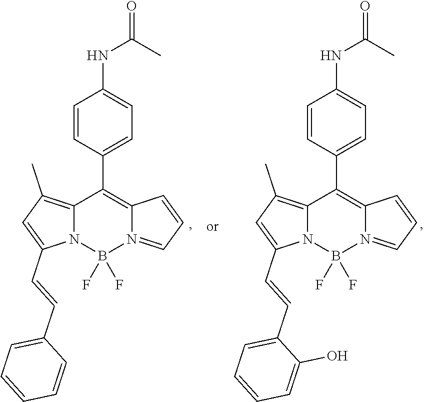

but is not necessarily limited thereto.

Another example embodiment provides a fluorescent probe for sensing changes in pH and/or viscosity of oil, comprising the compound represented by Chemical Formula 1 or the hydrate thereof:

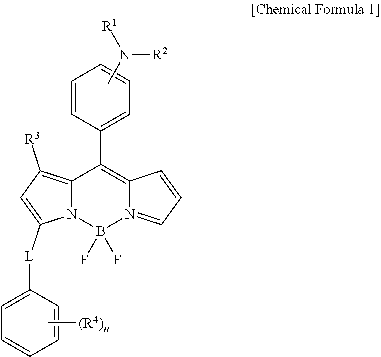

wherein
R¹ and R² are independently of each other —H, —C(O)H, C₁₋₁₀ alkyl, or C₁₋₁₀ alkylcarbonyl;
R³ is —H or C₁₋₅ alkyl;
R⁴ is independently of each other —H, —OH, —NH₂, C₁₋₈ alkyl, C₁₋₈ alkoxy, C₁₋₈ alkylamino, or C₁₋₈ alkylcarbonyloxy;
n is an integer of 1 to 3; and
L is C₂₋₁₀ alkenylene.

In an example embodiment, the alkenylene may comprise alkenylene directly connected to unsaturated carbon or alkenylene in which unsaturated carbon intervenes in and is connected to alkylene.

In an example embodiment, the “alkyl” comprises straight chain or branched chain alkyl, and also, those substituted by a substituent (for example, a halogen atom) which may be easily substituted by a person skilled in the art.

In an example embodiment, R¹ and R² may be independently of each other —H, —C(O) H, C₁₋₆ alkyl, C₁₋₆ alkyl, C₁₋₅ alkyl, C₁₋₃ alkyl, C₁₋₂ alkyl, —CH₃, C₁₋₈ alkylcarbonyl, C₁₋₆ alkylcarbonyl, C₁₋₅ alkylcarbonyl, C₁₋₃ alkylcarbonyl, C₁₋₂ alkylcarbonyl, or —C(O)CH₃.

In an example embodiment, R³ may be —H, C₁₋₅ alkyl, C₁₋₄ alkyl, C₁₋₃ alkyl, C₁₋₂ alkyl, or —CH₃.

In an example embodiment, R⁴ may be independently of each other —H, —OH, —NH₂, C₁₋₁₀ alkoxy, C₁₋₈ alkoxy, C₁₋₆ alkoxy, C₁₋₈ alkoxy, C₁₋₃ alkoxy, C₁₋₂ alkoxy, —OCH₃, C₁₋₁₀ alkylamino, C₁₋₈ alkylamino, C₁₋₆ alkylamino, C₁₋₅ alkylamino, C₁₋₃ alkylamino, C₁₋₂ alkylamino, or —NHCH₃.

In an example embodiment, n may be 1 or 2.

In an example embodiment, L may be C₂₋₅ alkenylene, C₂-4 alkenylene, C₂₋₃ alkenylene, or —CH═CH—.

In an example embodiment, when R⁴ is not —H, the compound may be ortho-, para-, or meta-substituted.

In an example embodiment, R¹ may be substituted at a meso-compound position, that is, at a para position. That is, the compound represented by Chemical Formula 1 may be a compound represented by the following Chemical Formula 2:

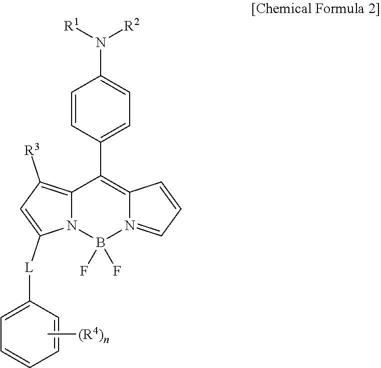

wherein R¹, R², R³, R⁴, n, and L are as defined above in Chemical Formula 1.

In an example embodiment, the compound represented by Chemical Formula 1 may be, specifically, for example,

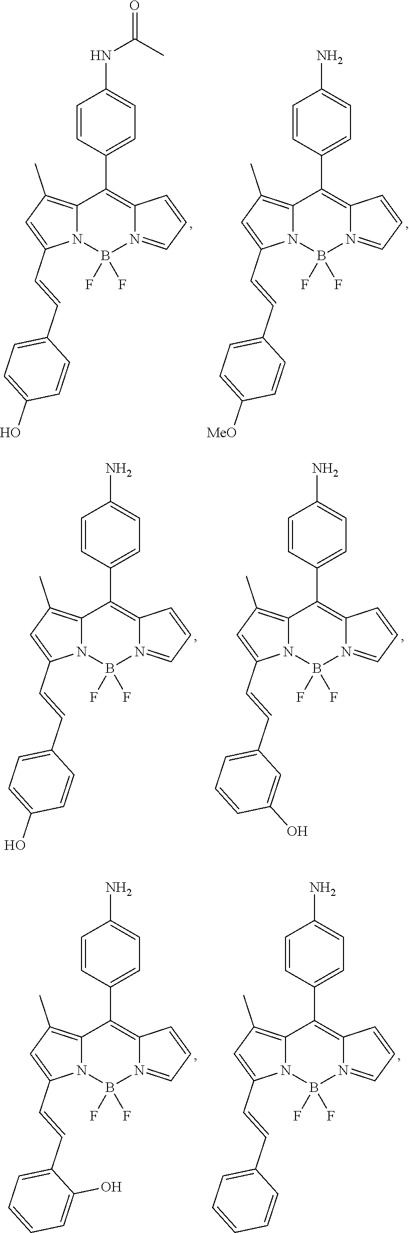

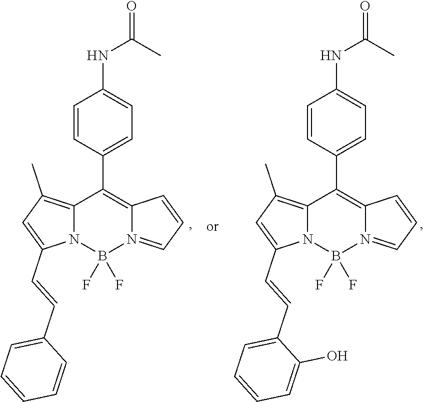

but is not necessarily limited thereto.

In an example embodiment, the compound shows excellent sensitivity to harmful oil by an aniline or an aniline derivative compound being substituted in a BODIPY compound. Specifically, the compound may release almost no fluorescence in fresh oil by an aniline or aniline derivative compound being substituted in the BODIPY compound, but release very high fluorescence in an oil after being cooked by heating and the like (that is, harmful oil), thereby sensing a harmful oil. In addition, since the compound and/or the fluorescent probe comprising the compound release(s) fluorescence in linear relationship to the amount of harmful oil, even a small amount of oil may be detected, and an amount of harmful oil may be quantitatively measured. In addition, since the compound and/or the fluorescent probe comprising the compound has an increased fluorescence intensity with increased cooking time, cooking time may be monitored.

In an example embodiment, since the compound and/or the fluorescent probe comprising the compound may be applied to various oils, for example, cooking oils extracted from various plants, they may be more appropriate for being commercialized in the field.

In an example embodiment, the compound and/or the fluorescent probe comprising the compound may sensitively react to pH and/or viscosity of oil, thereby sensing harmful oil. Specifically, oil (for example, cooking oil) has increased viscosity and decreased pH with heating by heat in the air, and as the viscosity of the compound according to an example embodiment and/or the fluorescent probe comprising the compound has increased viscosity, the fluorescence intensity is stronger, and/or as the pH is decreased, the fluorescence intensity is stronger, and thus, oil used in cooking may be sensed, using the fluorescent probe.

In an example embodiment, a harmful oil may refer to an oil heated by heat in the air, an oil used in cooking, an oil having decreased pH, or an oil having increased viscosity, as described above.

In an example embodiment, the oil may be, for example, cooking oil, but is not necessarily limited thereto.

An example embodiment provides a method of determining pH and/or viscosity of oil or sensing changes in the pH and/or viscosity of oil, using the fluorescent probe according to the example embodiment.

In an example embodiment, the method may comprise: mixing the fluorescent probe and the oil; and measuring a fluorescence intensity.

In an example embodiment, the method may comprise the following steps (methods): analyzing the measured fluorescence intensity to determine (predict) harmfulness of oil; and/or analyzing the measured fluorescence intensity to determine (predict) whether harmful oil is present; and/or analyzing the measured fluorescence intensity to determine (predict) quantitative amount of harmful oil; and/or analyzing the measured fluorescence intensity to determine (predict) cooking time; and/or analyzing the measured fluorescence intensity to determine (predict) pH and/or viscosity of oil.

The above determination and/or prediction may be performed by creating criteria for determination and/or prediction using various samples and following the criteria. In addition, when the method according to an example embodiment is used, waste oil may be sensed or detected.

In an example embodiment, the fluorescent probe is built into a bad oil sensing system (BOSS) as a portable platform, which is favorable for commercialization, and may be effectively applied to on-site cooking oil monitoring.

An example embodiment provides a kit for detecting pH and/or viscosity of oil, comprising the fluorescent probe according to the example embodiment.

The detection kit according to an example embodiment may be a kit which allows detection of a relative degree of pH and/or viscosity of oil. Otherwise, the detection kit according to an example embodiment may be a kit which allows determination or detection of a certain range of values of pH and/or viscosity of oil. By performing a predetermined number of repeated tests using the fluorescent probe according to an example embodiment, a correlation between the fluorescence intensity and the pH and/or viscosity value of oil is found to standardize the test results, and the values in the above range may be determined or detected using the standardized values. Therefore, an absolute or relative degree of the pH and/or viscosity of oil may be determined, using the detection kit according to an example embodiment.

In addition, changes in the pH and/or viscosity of oil may be detected or determined, using the detection kit according to an example embodiment.

The kit according to an example embodiment may be used as a kit for detecting or sensing waste oil. The waste oil refers to an oil having viscosity higher than a predetermined standard and/or pH lower than a predetermined standard.

An example embodiment provides a device for detecting pH and/or viscosity of oil, comprising the fluorescent probe according to an example embodiment.

The detection device according to an example embodiment may comprise the detection kit according to an example embodiment.

The detection device according to an example embodiment may be a device which allows detection of a relative degree of pH and/or viscosity of oil. Otherwise, the detection device according to an example embodiment may be a device which allows determination or detection of a certain range of values of pH and/or viscosity of oil. By performing a predetermined number of repeated tests using the fluorescent probe according to an example embodiment, a correlation between the fluorescence intensity and the pH and/or viscosity of oil is found to standardize the test results, and the values in the above range may be determined or detected using the standardized values. Therefore, an absolute or relative degree of the pH and/or viscosity of oil may be determined, using the detection device according to an example embodiment.

In addition, changes in the pH and/or viscosity of oil may be detected or determined, using the detection device according to an example embodiment.

The device according to an example embodiment may be used as a detection or sensing device of waste oil. The waste oil refers to an oil having viscosity higher than a predetermined standard and/or pH lower than a predetermined standard.

An example embodiment provides a system for monitoring changes in pH and/or viscosity of oil, comprising the fluorescent probe according to an example embodiment.

The monitoring system according to an example embodiment may monitor changes in pH and/or viscosity of oil in real time, using a fluorescence intensity. The monitoring system allows relative observation of changes in pH and/or viscosity by observing changes in a fluorescence intensity, and also allows observation of absolute numerical changes in pH and/or viscosity, using the standardized values. The monitoring system may comprise the fluorescent probe according to an example embodiment, and a kit or device comprising the fluorescent probe. Otherwise, the monitoring system may comprise a program comprising a formula for correlation between the established fluorescence intensity and pH and/or viscosity of oil, using the fluorescent probe according to an example embodiment.

### BEST MODE FOR CARRYING OUT THE INVENTION

Hereinafter, the examples and the experimental examples will be illustrated specifically in detail in the following. However, the examples and the experimental examples described later are only a partial illustration, and the technology described in the present specification is not construed as being limited thereto.

### Examples 1, 7, and 8

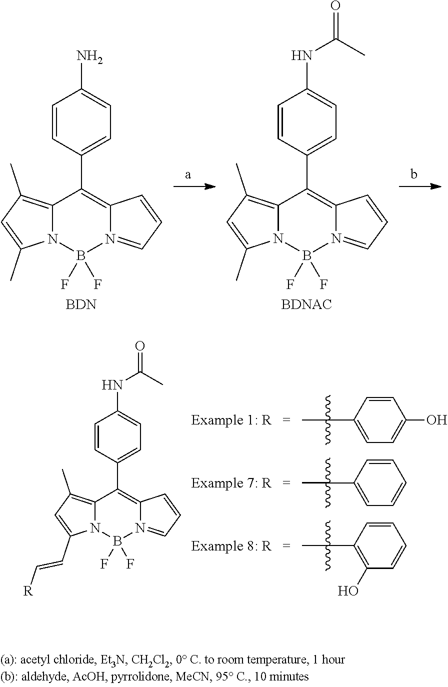

The compound of Example 1 was prepared according to the methods described in Literature 1 (H. Y. Kwon, X. Liu, E. G. Choi, J. Y. Lee, S. Y. Choi, J. Y. Kim, L. Wang, S. J. Park, B. Kim, Y. A. Lee, J. J. Kim, N. Y. Kang, Y. T. Chang, Angew. Chem. Int. Ed. 2019, 58, 8426-8431.) and Literature 2 (N. Y. Kang, S. C. Lee, S. J. Park, H. H. Ha, S. W. Yun, E. Kostromina, N. Gustavsson, Y. Ali, Y. Chandran, H. S. Chun, M. A. Bae, J. H. Ahn, W. Han, G. K. Radda, Y. T. Chang, Angew. Chem. Int. Ed. Engl. 2013, 52, 8557-8560.).

¹H NMR (500 MHz, Methanol-d₄) δ 7.77-7.73 (m, 2H), 7.62 (s, 1H), 7.52 (d, J=1.9 Hz, 1H), 7.52-7.49 (m, 3H), 7.36-7.32 (m, 2H), 6.90 (s, 1H), 6.87-6.83 (m, 2H), 6.41 (dd, J=3.9, 2.1 Hz, 1H), 6.40-6.37 (m, 1H), 2.19 (s, 3H), 1.64 (d, J=1.1 Hz, 3H). ¹³C NMR (126 MHz, Methanol-d₄) δ 170.47, 159.71, 159.24, 146.05, 140.83, 140.35, 139.88, 136.34, 134.80, 134.77, 130.15, 129.53, 129.50, 129.43, 127.61, 124.75, 119.27, 115.63, 115.12, 114.88, 22.55, 14.25.

The compound of Example 7 was also prepared according to the methods described in Literature 1 and Literature 2.

¹H NMR (500 MHz, Methylene Chloride-d₂) δ 7.77-7.70 (m, 4H), 7.68 (d, J=7.2 Hz, 2H), 7.50 (d, J=9.5 Hz, 2H), 7.46 (d, J=7.5 Hz, 2H), 7.43 (d, J=7.1 Hz, 1H), 7.40 (d, J=8.4 Hz, 2H), 6.84 (s, 1H), 6.52 (d, J=3.7 Hz, 1H), 6.47 (dd, J=3.8, 2.0 Hz, 1H), 2.23 (s, 3H), 1.71 (s, 3H). ¹³C NMR (126 MHz, Methylene Chloride-d₂) δ 168.31, 157.68, 146.20, 141.92, 139.62, 139.56, 138.05, 135.86, 135.15, 134.94, 129.85, 129.84, 129.31, 128.93, 127.79, 126.51, 119.17, 119.03, 118.39, 116.11, 24.49, 15.30.

The compound of Example 8 was prepared by the following method: 2—Hydroxybenzaldehyde (19.4 μL, 182.4 μmol) was added to a BDNAC solution (16.1 mg, 45.6 μmol) of 5 mL of an anhydrous ACN solvent, and then pyrrolidone (22.89 μL, 273.6 μmol) and AcOH (15.6 μL, 273.6 μmol) were added thereto. The solution was refluxed at 95° C. for 10 minutes and then cooled to room temperature, and the product was concentrated under vacuum and then purified with prep. HPLC to obtain the compound of Example 8 (11.4 mg, yield: 54.7%).

¹H NMR (850 MHz, DMSO-d₆) δ 10.38 (s, 1H), 10.21 (s, 1H), 7.82 (d, J=16.4 Hz, 1H), 7.80-7.75 (m, 3H), 7.71 (s, 1H), 7.51 (d, J=7.6 Hz, 1H), 7.41 (d, J=8.2 Hz, 2H), 7.25 (t, J=7.5 Hz, 1H), 7.15 (s, 1H), 6.94 (d, J=8.1 Hz, 1H), 6.90 (t, J=7.4 Hz, 1H), 6.45 (s, 1H), 6.40 (d, J=3.3 Hz, 1H), 2.10 (s, 3H), 1.63 (s, 3H). ¹³C NMR (214 MHz, DMSO-d₆) δ 168.69, 158.66, 156.82, 145.78, 140.99, 140.59, 137.27, 137.07, 134.34, 134.27, 131.42, 129.65, 128.78, 127.64, 125.39, 122.46, 119.82, 119.67, 118.46, 118.01, 116.35, 116.06, 24.10, 15.10.

### Examples 2 to 6

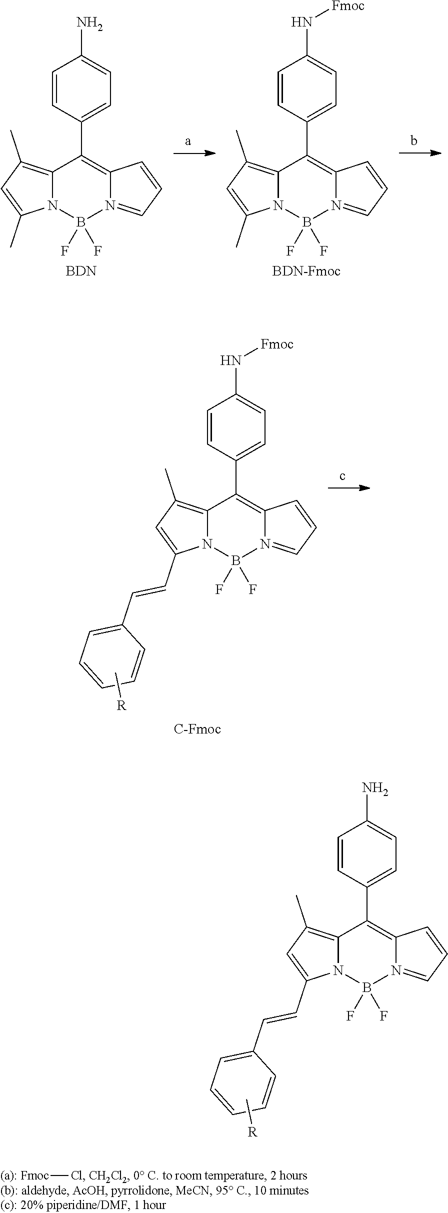

Fmoc-Cl (1.5 equiv.) was added to a BDN solution (1 equiv.) of a DCM solvent at 0° C., stirring was performed at 0° C. for 10 minutes, and then stirring was performed at room temperature for 2 hours. The product was concentrated under vacuum, and then normal phase silica column chromatography was used to obtain BDN-Fmoc. Next, aldehyde (4 equiv.) was added to BDN-Fmoc (15 mg, 1 equiv.) of an anhydrous ACN solvent, and then pyrrolidone (6 equiv.) and AcOH (6 equiv.) were added thereto. The solution was refluxed at 95° C. for 10 minutes and then cooled to room temperature, and the product was concentrated under vacuum and then purified with prep. HPLC to obtain a C-Fmoc compound. Next, 20% piperidine/DMC (v/v) was added to the C-Fmoc, stirring was performed for 1 hour, and the product was concentrated under vacuum and then purified with prep. HPLC to obtain the compounds of Examples 2 to 6.

Example 2 (7.8 mg, yield: 64.7%):

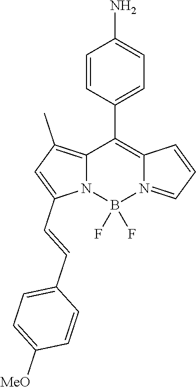

¹H NMR (500 MHz, DMSO-d₆) δ 7.72 (d, J=16.3 Hz, 1H), 7.67 (s, 1H), 7.60 (d, J=8.8 Hz, 2H), 7.40 (d, J=16.3 Hz, 1H), 7.23 (d, J=8.4 Hz, 2H), 7.12 (s, 1H), 7.06 (d, J=8.8 Hz, 2H), 6.84 (d, J=8.3 Hz, 2H), 6.49 (dd, J=3.9, 1.3 Hz, 1H), 6.46 (dd, J=3.9, 2.1 Hz, 1H), 3.82 (s, 3H), 1.72 (s, 3H). ¹³C NMR (126 MHz, DMSO-d₆) δ 160.94, 157.08, 145.27, 142.48, 139.90, 136.65, 134.33, 134.13, 130.80, 129.31, 128.35, 125.38, 119.65, 115.90, 115.37, 114.96, 114.81, 55.42, 15.58.

Example 3 (3.0 mg, yield: 25.7%):

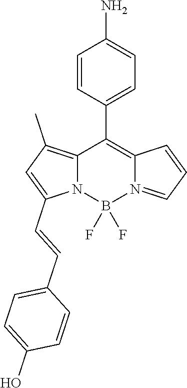

¹H NMR (500 MHz, Methanol-d₄) δ 7.60 (s, 1H), 7.53-7.49 (m, 4H), 7.35-7.30 (m, 2H), 7.14-7.11 (m, 2H), 6.93 (s, 1H), 6.86-6.83 (m, 2H), 6.40 (d, J=1.9 Hz, 2H), 1.69 (s, 3H). ¹³C NMR (126 MHz, Methanol-d₄) δ 161.08, 160.35, 147.25, 142.23, 141.95, 137.62, 136.15, 131.93, 130.82, 129.04, 126.10, 120.58, 118.91, 117.03, 116.95, 116.45, 116.34, 15.76.

Example 4 (5.1 mg, yield: 43.8%):

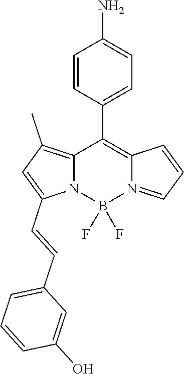

¹H NMR (500 MHz, Methanol-d₄) δ 7.63 (t, J=8.2 Hz, 2H), 7.44 (d, J=16.3 Hz, 1H), 7.23 (t, J=8.1 Hz, 1H), 7.15-7.11 (m, 2H), 7.08 (dd, J=4.1, 2.3 Hz, 2H), 6.91 (s, 1H), 6.81 (ddt, J=7.4, 3.0, 1.6 Hz, 3H), 6.56 (d, J=3.5 Hz, 1H), 6.42 (dd, J=3.9, 2.1 Hz, 1H), 1.77 (s, 3H). ¹³C NMR (126 MHz, Methanol-d₄) δ 159.11, 158.35, 151.42, 147.21, 145.34, 140.36, 138.94, 138.43, 136.64, 136.08, 131.73, 131.01, 127.46, 123.67, 120.64, 120.17, 119.56, 118.03, 116.79, 115.42, 114.49, 15.93.

Example 5 (3.0 mg, yield: 25.7%):

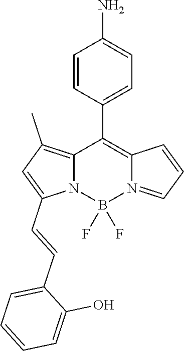

¹H NMR (850 MHz, DMSO-d₆) δ 10.29 (s, 1H), 7.78-7.72 (m, 2H), 7.65 (s, 1H), 7.51-7.49 (m, 1H), 7.25-7.22 (m, 1H), 7.14 (d, J=8.3 Hz, 2H), 7.11 (s, 1H), 6.93 (d, J=8.1 Hz, 1H), 6.90 (t, J=7.4 Hz, 1H), 6.70 (d, J=8.4 Hz, 2H), 6.53 (d, J=3.6 Hz, 1H), 6.46 (dd, J=3.8, 2.1 Hz, 1H), 5.70 (s, 2H), 1.76 (s, 3H). ¹³C NMR (214 MHz, DMSO-d₆) 157.22, 156.59, 150.82, 145.25, 143.26, 136.51, 135.66, 134.39, 134.14, 131.10, 130.78, 128.42, 125.43, 122.61, 120.01, 119.64, 119.32, 118.17, 116.30, 115.79, 113.22, 15.60.

Example 6 (5.6 mg, yield: 49.9%):

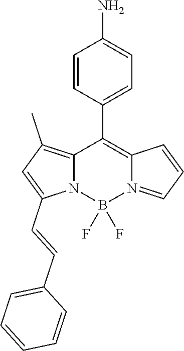

¹H NMR (500 MHz, DMSO-d₆) δ 7.75-7.69 (m, 2H), 7.63 (d, J=7.4 Hz, 2H), 7.54 (d, J=16.4 Hz, 1H), 7.48 (t, J=7.5 Hz, 2H), 7.45-7.39 (m, 1H), 7.18-7.12 (m, 3H), 6.73-6.68 (m, 2H), 6.62-6.58 (m, 1H), 6.49 (dd, J=3.9, 2.1 Hz, 1H), 5.75 (s, 2H), 1.78 (s, 3H). ¹³C NMR (126 MHz, DMSO-d₆) δ 155.64, 151.03, 145.04, 144.30, 139.02, 137.40, 135.74, 134.52, 133.97, 130.91, 129.80, 129.19, 127.34, 126.35, 119.86, 119.46, 117.85, 116.24, 113.22, 15.67.

The compounds of Examples 1 to 8 are summarized in the following Table 1:

TABLE 1

| No. | Chemical Formula |
|---|---|
| 1 | 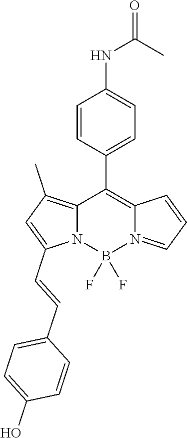 |
| 2 | 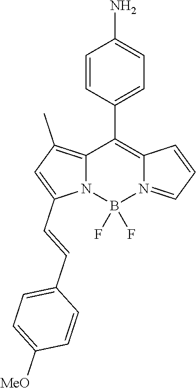 |
| 3 | 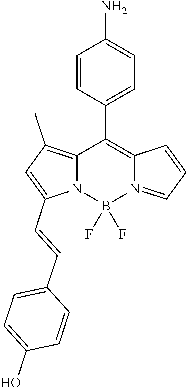 |
| 4 | 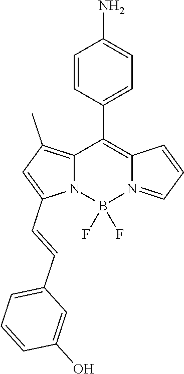 |
| 5 | 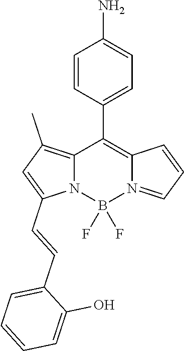 |
| 6 | 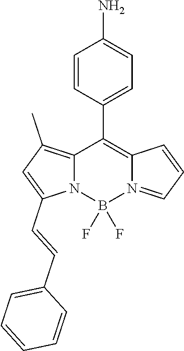 |
| 7 | 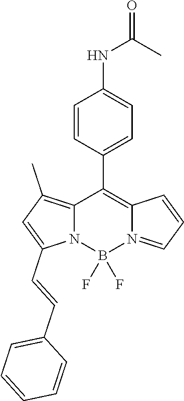 |
| 8 | 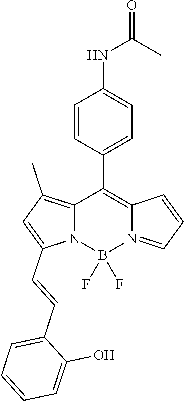 |

### Comparative Example 1

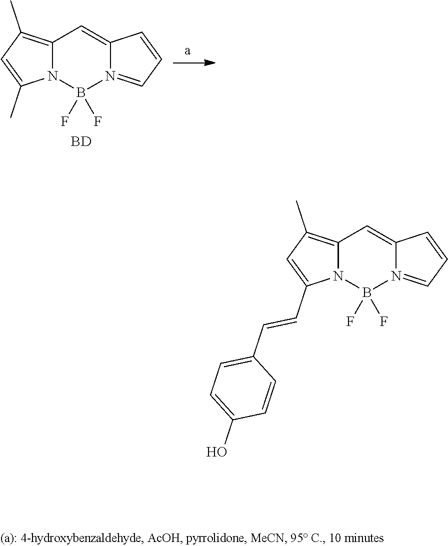

4-Hydroxybenzaldehyde (33.3 mg, 272.8 μmol) was added to 5 mL of a BODIPY (BD) solution (15 mg, 68.2 μmol) of an hydrous ACN solvent, and pyrrolidone (34.1 μL, 409.2 μmol) and AcOH (23.4 μL, 409.2 μmol) were added thereto. The solution was refluxed at 95° C. for 10 minutes and then cooled to room temperature, and the product was concentrated under vacuum and then purified with prep. HPLC to obtain the compound of Comparative Example 1 (17.3 mg, yield: 78.3%).

¹H NMR (500 MHz, Acetonitrile-d₃) δ 7.71 (s, 1H), 7.69-7.61 (m, 3H), 7.49 (d, J=16.5 Hz, 2H), 7.09 (d, J=3.8 Hz, 1H), 7.01-6.97 (m, 3H), 6.57 (dd, J=3.8, 2.1 Hz, 1H), 2.41 (s, 3H). ¹³C NMR (126 MHz, Acetonitrile-d₃) δ 160.95, 160.24, 147.09, 142.37, 139.13, 138.17, 133.87, 130.86, 128.86, 126.20, 124.20, 117.06, 116.90, 116.88, 115.89, 115.87, 115.85, 11.65.

### Preparation of Oil Sample

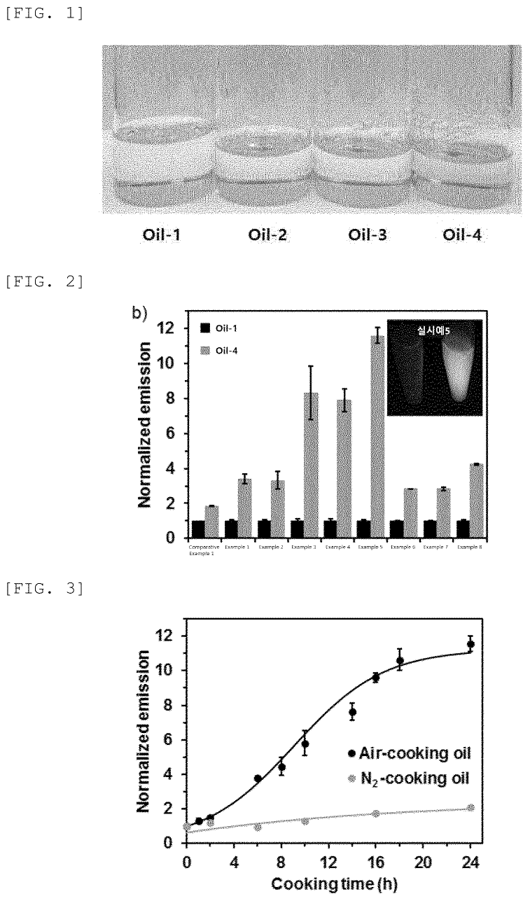

The following oil (cooking oil) was prepared for the experiment. The experiment was performed by continuously stirring oil, using an oil bath at 190° C. in order to create an environment similar to real cooking conditions. As the oil, a fresh oil (cooking oilOil-1) prepared for the experiment, a 24 h-air-bubbling oil (Oil-2) which was continuously stirred while injecting air without heating, a 24 h-N₂-cooking oil (Oil-3) which was heated after filling a vial having a cap with a nitrogen (N₂) gas, and a 24 h-air-cooking oil (Oil-4) which was heated while injecting air were prepared. As a result of treating the oils under each condition for 24 hours, Oil-1 to Oil-3 did not show large color changes, but Oil-4 became a yellow sticky fluid, which was a harmful oil contaminated with various harmful materials after cooking (FIG. 1).

### <Experimental Example 1> Analysis of Fluorescence Reaction

### 1-1. Analysis of Oil-1 to Oil-4

In order to analyze the fluorescence reactions of the compounds prepared in the examples and the comparative example with the fresh oil and the 24 h-air-cooking oil, 10 μM of the compounds of the examples and the comparative example were added to a 1 mM cetyltrimethylammonium bromide (CTAB) solution to which 5 v/v % of Oil-1 and Oil-4 were added, respectively, and their fluorescence intensities were measured at λₑₓ=530 nm. The results are shown in FIG. 2. As a result, the compound of Comparative Example 1 containing no meso-aniline group had a significantly low fluorescence intensity to Oil-4 as compared with the compounds of the examples. However, the compounds of Examples 1 to 8 had a very increased fluorescence intensity when reacted with Oil-4 as compared with Oil-1, and in particular, the compounds of Examples 3 to 5 had a fluorescence intensity increased by about 8 times when reacted with Oil-4 as compared with Oil-1. Thus, it was confirmed that the harmful oil was able to be easily sensed using the compounds of the examples.

### 1-2. Analysis of Oil Depending on Cooking Time

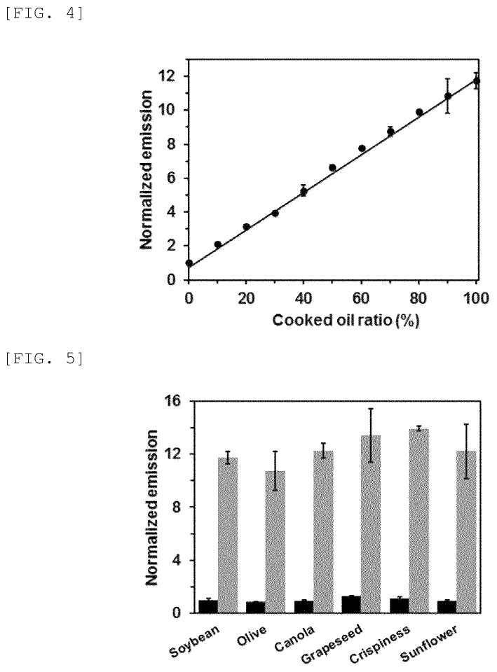

In order to confirm whether a cooking degree of oil may be monitored using the compounds of the examples, fluorescence reactions with oils used during various cooking times were analyzed using the compound of Example 5. The results are shown in FIG. 3. As a result of comparing cooking with oil while supplying air and cooking with oil under nitrogen gas, it was confirmed that the fluorescence intensities of the compounds of the examples were increased depending on the cooking time when cooking with oil while supplying air. In particular, when the cooking time had passed about 1 hour, the fluorescence intensity already increased by about 1.5 times, and thus, the detection sensitivity was confirmed to be very high. Thus, it was confirmed that the compounds of the examples allowed rapid sensing of harmful oil with high sensitivity.

### 1-3. Analysis by Amount of Oil

When the fresh oil is mixed with a small amount of harmful oil, sensing using a chemical indicator may be difficult. Thus, in order to confirm whether the harmful oil may be sensed using the compounds of the examples even when a small amount of harmful oil was mixed with the fresh oil, a fluorescence intensity was analyzed using mixed oils of Oil-1 and Oil-4 at each ratio of Oil-4 of 0% to 100% and the compound of Example 5. The results are shown in FIG. 4. As a result, the fluorescence intensity of the compound of Example 5 had a linear correlation for the mixed ratio. Therefore, it was found that when the compound of Example 5 was used, the harmful oil was able to be sensed with high sensitivity even when the harmful oil was mixed in a small amount, and also, the mixed ratio of the harmful oil was able to be quantified.

### 1-4. Analysis of Fluorescence Reaction for Various Cooking Oils

Since oils extracted from various plants have various components, in order to confirm whether the compounds of the examples react with those cooking oils, the experiment was performed using soybean oil (Soybean, 100%, OTTOGI Corp.), olive oil (Olive, 100%, CJ CHEILJEDANG CORP.), canola oil (Canola, 100%, CJ CHEILJEDANG CORP.), grapeseed oil (Grapeseed, 100%, CJ CHEILJEDANG CORP.), crispy cooking oil (Crispiness, 50% of canola oil and 49.8% of soybean oil, CJ CHEILJEDANG CORP.), and sunflower oil (Sunflower, 100%, HAEPYO), and the cooking oils were prepared as fresh oils before cooking (black bars in FIG. 5) and cooking oils heated for 24 hours while injecting air (blue bars in FIG. 5) and likewise, the fluorescence intensity was analyzed using the compound of Example 5. The results are shown in FIG. 5. As a result, the compound of Example 5 showed a very low fluorescence intensity to fresh cooking oil regardless of the type of cooking oils, and showed about 11 times to about 4 times higher fluorescence intensity to the harmful cooking oil heated for 24 hours.

### <Experimental Example 2> Analysis of Reactivity to pH and Viscosity

Various complicated reactions occur continuously during a cooking process, and crosslinking of unsaturated aliphatic acid may be accelerated by oxidation and polymerization by oxygen. In addition, when the cooking oil is exposed to heat, decomposition of oil is promoted, so that the concentration of free fatty acids (FFA) may be increased to further acidify oil. Therefore, as the more the compound is sensitive to pH and viscosity, the higher the reactivity to cooking oil is, and thus, the reactivities of the viscosity and pH of the compounds of the examples were evaluated. Viscosity sensitivity (x) was calculated using the following equation:

log FI=C+xlog η (FI is an emission maxima, and η is viscosity.)

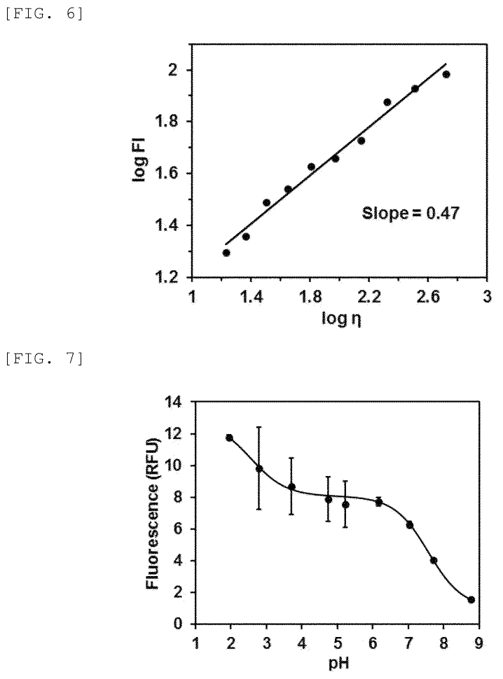

In addition, the reactivity to pH was determined by creating various pH environments using a 20 mM phosphoric acid buffer solution in a 1 mM CTAB solvent, adding 10 μM of the compound of Example 5, and measuring the fluorescence intensity at λₑₓ=530 nm. The results were shown in FIGS. 6 and 7. Thus, it was confirmed therefrom that since the compound of Example 5 sensitively reacted under the viscosity and pH conditions, it may sense cooking oil exposed to heat during a cooking process with high sensitivity.

Next, relative contribution of viscosity and pH to the reaction of the compound of Example 5 was analyzed. First, the viscosity-insensitive/pH-sensitive compound of the following Chemical Formula F1 was prepared:

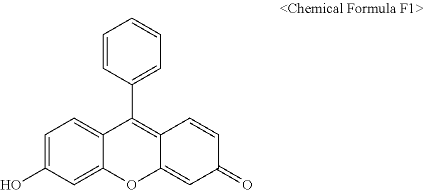 [SMILES: O=c1ccc2c(-c3ccccc3)c3ccc(O)cc3oc-2c1]

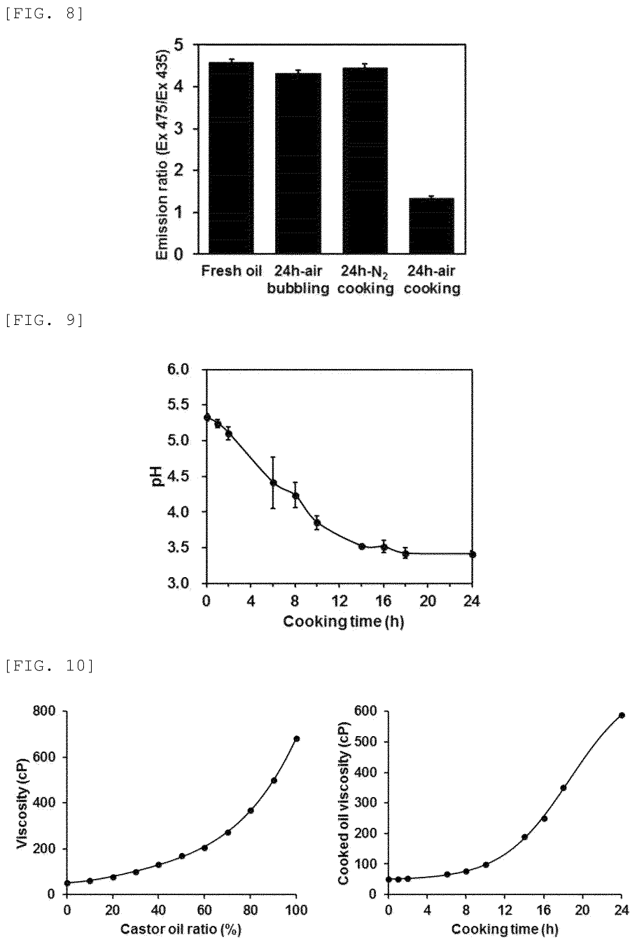

Next, in order to quantify the pH of the cooking oil emulsion in a CTAB solution, 10 μM of F1 was added, and then a release ratio (release at 475 nm/release at 435 nm) was analyzed (FIG. 8). As a result, the release ratio of F1 was decreased from about 4.58 to 1.35, after being cooked for 24 hours in the air. Thus, as a result of estimating the pH of the oil-CTAB emulsion, it was confirmed that the pH was decreased from about 5.33 to 3.41 (FIG. 9). Therefore, it was found that the cooking oil cooked in the air for 24 hours was much more acidic than fresh cooking oil, and as heating was continued in the air, the pH of the oil emulsion was decreased and FFA was formed.

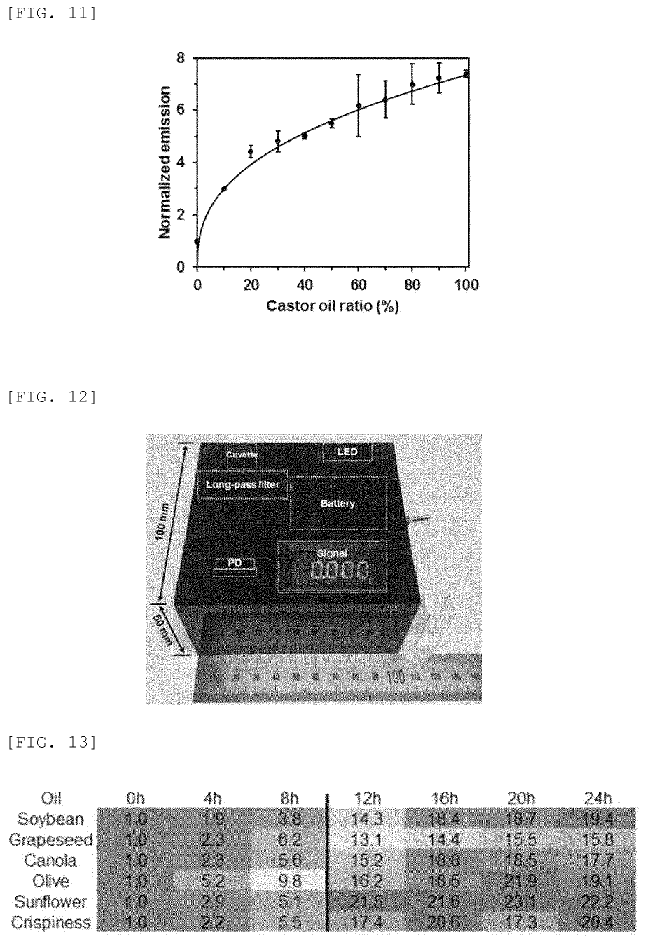

Next, in order to exclude the pH effect and quantify the effect on viscosity, the experiment was performed as follows. First, a mixture of fresh cooking oil and castor oil was prepared by a viscosity model system. As a result of measuring viscosity depending on a mixing ratio of the castor oil in the mixture using a viscometer, the viscosity was increased with the increased ratio of the castor oil. In addition, the viscosity of the mixture was increased with increased cooking time (FIG. 10). In this regard, as a result of analyzing the fluorescence intensity of the compound of Example 5 depending on the mixing ratio of the castor oil in the mixture, the fluorescence intensity was increased with the increased ratio of the castor oil, and as a result of analyzing the fluorescence intensity of the compound of Example 5 depending on cooking time, the fluorescence intensity was increased with increased cooking time (FIG. 11). Thus, it was confirmed that viscosity was a very important factor in sensing harmful cooking oil.

Thus, as a result of analyzing the reaction of the compound of Example 5 to pH and viscosity through the experiment, it was found that viscosity plays an important role in the initial reaction step, and the contribution of pH was increased with increased cooking time (FIG. 12).

Meanwhile, the factor of increased viscosity of cooking oil is expected to be polymerization of unsaturated aliphatic acid, and the pH decrease factor is expected to be decomposition of triglyceride after cooking.

### <Experimental Example 3> Building of Bad Oil Sensing System (BOSS)

In order to confirm whether rapid and quantitative detection was allowed using the compounds of the examples, a bad oil sensing system (BOSS) was built as a portable platform. BOSS comprised a light source, a filter module, a sensing module, and a signal display module, and the results of detecting harmful oil was reflected on the number indicated as a signal (FIG. 12). As a result of measuring various cooking oils [soybean oil (Soybean, 100%, OTTOGI Corp.), olive oil (Olive, 100%, CJ CHEILJEDANG CORP.), canola oil (Canola, 100%, CJ CHEILJEDANG CORP.), grapeseed oil (Grapeseed, 100%, CJ CHEILJEDANG CORP.), crispy cooking oil (Crispiness, 50% of canola oil and 49.8% of soybean oil, CJ CHEILJEDANG CORP.), and sunflower oil (Sunflower, 100%, HAEPYO)] using BOSS while heating for 24 hours, a map was produced as shown in FIG. 13, and generally all cooking oils showed significantly high fluorescence after cooking for 12 hours (FIG. 13). Thus, high potential for commercialization and on-site cooling oil monitoring application was confirmed by confirming universal applicability of portable BOSS.

Hereinabove, though the present disclosure has been described in detail by the preferred examples and experimental examples, the scope of the present disclosure is not limited to specific examples, and should be construed by the appended claims. In addition, it should be understood by a person skilled in the art that many modifications and variations are possible without departing from the scope of the present disclosure.

1. A compound represented by the following Chemical Formula 1 or a hydrate thereof: 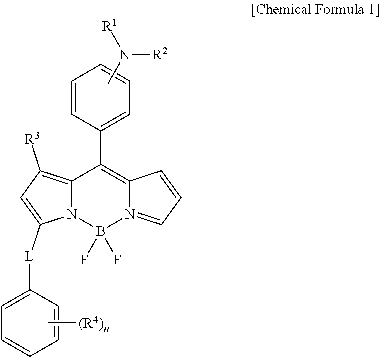
wherein
R¹ and R² are independently of each other —H, —C(O)H, C₁₋₁₀ alkyl, or C₁₋₁₀ alkylcarbonyl;
R³ is —H or C₁₋₅ alkyl;
R⁴ is independently of each other —H, —OH, —NH₂, C₁₋₈ alkyl, C₁₋₈ alkoxy, C₁₋₈ alkylamino, or C₁₋₈ alkylcarbonyloxy;
n is an integer of 1 to 3; and
L is C₂₋₁₀ alkenylene.

2. The compound or the hydrate thereof of claim 1, wherein R¹ and R² are independently of each other —H, —C(O)H, C₁₋₅ alkyl, or C₁₋₅ alkylcarbonyl.

3. The compound or the hydrate thereof of claim 1, wherein R¹ and R² are independently of each other —H or —C(O)CH₃.

4. The compound or the hydrate thereof of claim 1,
wherein R⁴ is independently of each other —H, —OH, —NH₂, C₁₋₅ alkoxy, or C₁₋₅ alkylamino;
n is 1 or 2; and
L is C₂₋₅ alkenylene.

5. The compound or the hydrate thereof of claim 1, wherein R⁴ is independently of each other —H, —OH, or —OCH₃.

6. The compound or the hydrate thereof of claim 1, wherein the compound represented by Chemical Formula 1 is

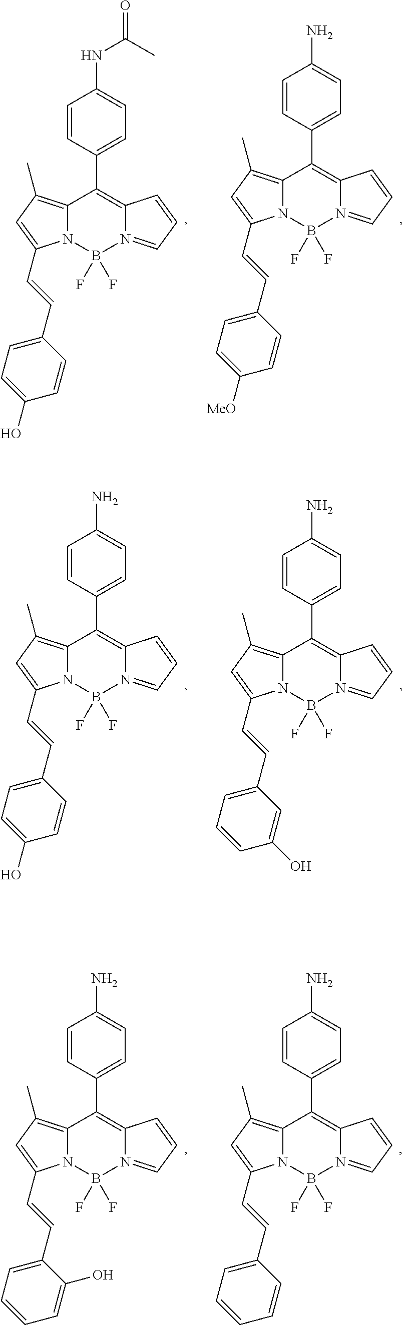

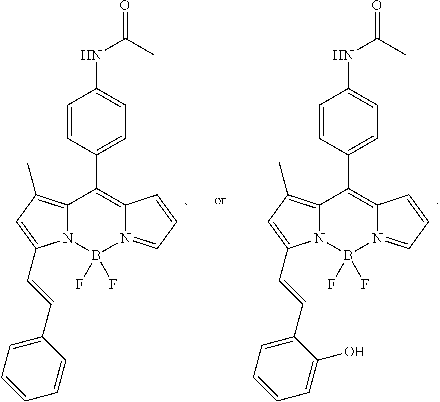

7. A fluorescent probe for sensing changes in pH and viscosity of oil, comprising a compound represented by the following Chemical Formula 1 or a hydrate thereof: 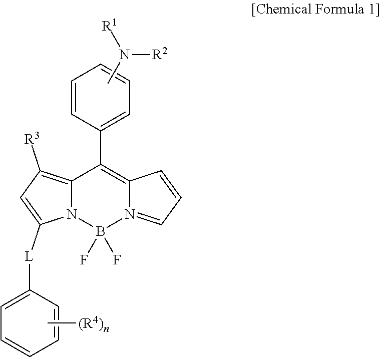
wherein
R¹ and R² are independently of each other —H, —C(O)H, C₁₋₁₀ alkyl, or C₁₋₁₀ alkylcarbonyl;
R³ is —H or C₁₋₅ alkyl;
R⁴ is independently of each other —H, —OH, —NH₂, C₁₋₈ alkyl, C₁₋₈ alkoxy, C₁₋₈ alkylamino, or C₁₋₈ alkylcarbonyloxy;
n is an integer of 1 to 3; and
L is C₂₋₁₀ alkenylene.

8. The fluorescent probe for sensing changes in pH and viscosity of oil of claim 7, wherein R¹ and R² are independently of each other —H, —C(O)H, C₁₋₅ alkyl, or C₁₋₅ alkylcarbonyl.

9. The fluorescent probe for sensing changes in pH and viscosity of oil of claim 7, wherein R¹ and R² are independently of each other —H or —C(O)CH₃.

10. The fluorescent probe for sensing changes in pH and viscosity of oil of claim 7,
wherein R⁴ is independently of each other —H, —OH, —NH₂, C₁₋₅ alkoxy, or C₁₋₅ alkylamino;
n is 1 or 2; and
L is C₂₋₅ alkenylene.

11. The fluorescent probe for sensing changes in pH and viscosity of oil of claim 7, wherein R⁴ is independently of each other —H, —OH, or —OCH₃.

12. The fluorescent probe for sensing changes in pH and viscosity of oil of claim 7, wherein the compound represented by Chemical Formula 1 is

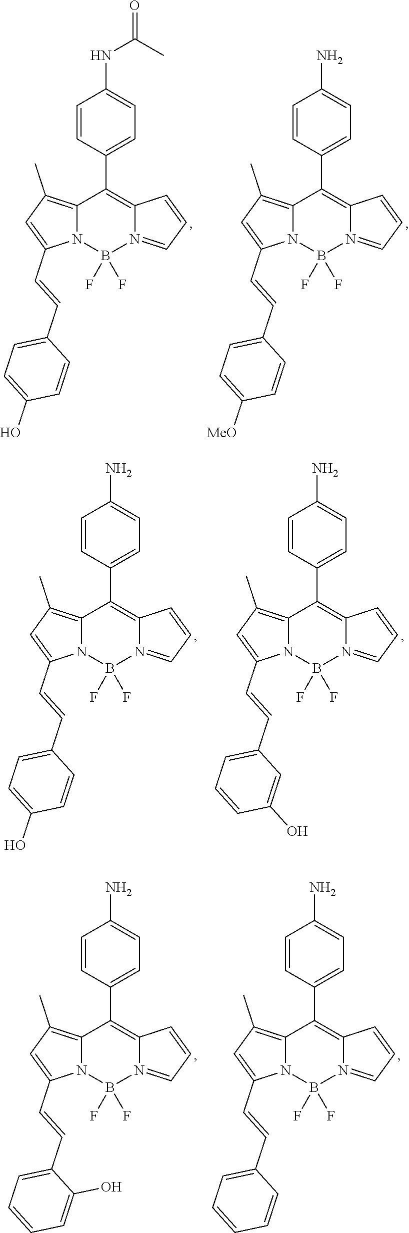

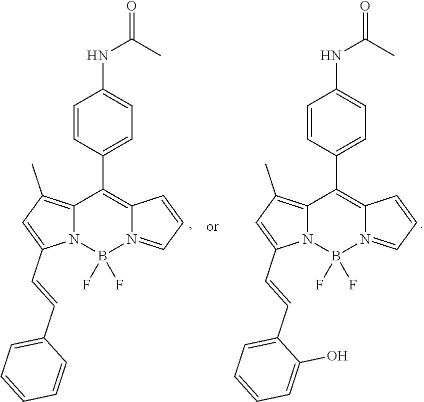

13. The fluorescent probe for sensing changes in pH and viscosity of oil of claim 7, wherein the oil is a cooking oil.

14. A method of sensing changes in pH and viscosity of oil, using the fluorescent probe for sensing changes in pH and viscosity of oil according to claim 7.

15. The method of sensing changes in pH and viscosity of oil of claim 14, wherein the method comprises:
mixing the fluorescent probe and oil; and
measuring a fluorescence intensity.

16. A kit for detecting pH and viscosity of oil, comprising the fluorescent probe for sensing changes in pH and viscosity of oil according to claim 7.

17. A device for detecting pH and viscosity of oil, comprising the fluorescent probe for sensing changes in pH and viscosity of oil according to claim 7.

18. A system for monitoring changes in pH and viscosity of oil, comprising the fluorescent probe for sensing changes in pH and viscosity of oil according to claim 7.
